SEQUENCE LISTING (2 sequences)

SEQ ID NO: 1 - DNA, 33 bp, Artificial Sequence
  Description of Artificial Sequence synthesized oligonucleotide primer
cgtcaaggttccccggatctagagcaaaactcc

SEQ ID NO: 2 - DNA, 34 bp, Artificial Sequence
  Description of Artificial Sequence synthesized oligonucleotide primer
ccgcttgtcacctctagagtgtggggttcacgac

(19) United States
(12) Patent Application Publication
(10) Pub. No.: US 2003/0167015 A1
(43) Pub. Date: Sep. 4, 2003

(54) Animal model of atrial fibrillation and screening and therapeutic methods relating thereto

(76) Inventors: Dan M. Roden, Nashville, TN (US); Sabina Kupershmidt, Nashville, TN (US)

Correspondence Address: JENKINS & WILSON, PA, 3100 TOWER BLVD, SUITE 1400, DURHAM, NC, 27707, US

(21) Appl. No.: 09/877,586

(22) Filed: Jun. 8, 2001

Related U.S. Application Data
(60) Provisional application No. 60/210,695, filed on Jun. 9, 2000.

(51) Int. Cl. A61B 5/046

(52) U.S. Cl. 600/518

(57) ABSTRACT

An animal model of atrial fibrillation and a method of identifying an animal exhibiting an atrial fibrillation phenotype. The method includes providing an animal having a minK (−/−) genotype; recording electrocardiograms from the animal over an extended time period; and identifying an atrial fibrillation pattern in the electrocardiograms to thereby identify the animal as exhibiting an atrial fibrillation phenotype. A method of screening candidate substances for activity in the treatment of atrial fibrillation using an animal model is also disclosed, as is a method of treating atrial fibrillation using a candidate compound identified as having activity in the treatment of atrial fibrillation.

### CROSS REFERENCE TO RELATED APPLICATIONS

This application is based on and claims priority to U.S. Provisional Application Serial No. 60/210,695, filed Jun. 9, 2000, the entire contents of which are herein incorporated by reference.

### GRANT STATEMENT

This work was supported by one or more of the following United States Public Health Service grants: HL46681, HL49989, HL03727, HL52813 and CA68485. Thus, the U.S. Government has certain rights in the invention.

### TECHNICAL FIELD

The present invention pertains generally to an animal model for atrial fibrillation. More particularly, the present invention pertains to a minK (−/−) animal model exhibiting an atrial fibrillation phenotype, and to screening and therapeutic methods relating to the animal model.

### BACKGROUND ART

Atrial fibrillation is a common health problem in the United States, affecting 2-5 million individuals. Symptoms may range from none to palpitations to exercise limitation due to uncontrolled rates and/or development of decreased cardiac contractility. In addition, atrial fibrillation is a major risk factor for stroke. The age distribution for atrial fibrillation is bell-shaped with a peak at approximately 75 years. The aging of the American population indicates that the incidence of this problem, and its attendant public health burden, is likely to rise substantially over the next decade or two, as the “baby boom” generation enters the age at which atrial fibrillation is especially common.

Atrial fibrillation is usually a recurrent problem. Patients often start by having brief episodes of paroxysmal atrial fibrillation, i.e. episodes that are self-terminating. These then become longer over time, and eventually require treatment both because of symptoms and to prevent stroke. In some patients, atrial fibrillation becomes permanent, i.e. efforts to return normal rhythm are unsuccessful, and rate control medications and anticoagulation medication are used.

Current therapeutic strategies in atrial fibrillation include drug therapies designed to prevent arrhythmia recurrence as well as ancillary therapies, such as control of ventricular response during atrial fibrillation. Anticoagulation is routinely undertaken and has been shown to decrease the incidence of stroke. Available drugs to maintain sinus rhythm are not completely effective and frequently produce side-effects, ranging from nuisance to the life-threatening (including forms of life-threatening arrhythmias). Thus, the problem is major, the therapies are only partially effective at best; and atrial fibrillation represents a major unmet medical need. The development of new drugs to maintain normal rhythm in atrial fibrillation would be a major therapeutic advance.

Studies in experimental animals as well as in humans suggest that atrial fibrillation may have a number of etiologies. Currently available experimental models include rapid atrial pacing in dogs or other large animals (which after 2-4 weeks does promote atrial fibrillation) and/or the application of action potential-modifying chemicals directly to the atrium, followed by atrial pacing. There is no currently available animal model in which episodes of atrial fibrillation occur spontaneously. Thus, the development of an animal model of atrial fibrillation represents a long-felt and continuing need for testing of new drug therapies for this condition.

### SUMMARY OF THE INVENTION

A method of identifying an animal exhibiting an atrial fibrillation phenotype. The method comprises providing an animal having an minK (−/−) genotype; recording electrocardiograms from the animal over an extended time period; and identifying an atrial fibrillation pattern in the electrocardiograms to thereby identify the animal as exhibiting an atrial fibrillation phenotype. An animal model of atrial fibrillation is thus also disclosed.

A method for screening candidate substances for an ability to modulate atrial fibrillation is also disclosed. The method comprises providing an animal model having an atrial fibrillation phenotype and a minK (−/−) genotype; administering a candidate compound to the animal model; and measuring atrial fibrillation in the animal model to thereby determine the ability of the candidate substance to modulate atrial fibrillation.

A method of modulating atrial fibrillation in a vertebrate is also disclosed. The method comprises administering to a vertebrate in need thereof an effective amount of a substance capable of modulating atrial fibrillation in the vertebrate, whereby modulation of atrial fibrillation is accomplished.

Accordingly, it is an object of the present invention to provide a novel animal model of atrial fibrillation, and to provide screening and therapeutic methods related thereto. This and other objects are achieved in whole or in part by the present invention.

An object of the invention having been stated hereinabove, other objects will become evident as the description proceeds when taken in connection with the accompanying Laboratory Examples as best described herein below.

### DETAILED DESCRIPTION OF THE INVENTION

The present invention pertains to an animal model of atrial fibrillation and to a method for making the animal model. In a preferred embodiment, the animal comprises a minK (−/−) animal that has been screened for episodes of paroxysmal atrial fibrillation in accordance with techniques disclosed herein.

The present invention also pertains to a method of screening candidate substances for activity in the treatment of atrial fibrillation using the animal model. The present invention also pertains to a method of treating atrial fibrillation using a candidate compound identified as having activity in the treatment of atrial fibrillation using the screening method and animal model of the present invention.

A. General Considerations

The minK gene was identified by the technique of “expression cloning” from a rat kidney cDNA library (Takumi, T., et al., (1988) Science 242:1042-5) as a probable potassium channel sub-unit. Subsequently, expression of the minK gene was detected in heart, and in some biological systems (such as Xenopus oocytes) expression of minK resulted in a slowly-activating delayed rectifier potassium current similar to the current I_{Ks} observed in certain cardiac myocytes. However, in other biological systems, notable mammalian cells, expression of minK did not result in any potassium current. Thus, the role of minK expression in cardiac biology was uncertain. Honoré, E. et al., EMBO. J. (1991) 10:2805-2811.

A series of experiments to delete the minK gene in mice, using the technique of homologous recombination, were undertaken by the present applicants. The targeting vector used to construct the mice included a bacterial reporter gene, LacZ, in the minK coding locus. LacZ expression is readily detected as blue staining, using simple biochemical methods. In the resulting mice, expression of LacZ was directed by authentic minK regulatory elements; thus, blue staining of cells in the genetically-modified mice indicates cells in which minK would ordinarily be expressed. Since undertaking the construction of the genetically modified animal, it has been observed that minK is actually a sub-unit that modulates expression or function of other potassium channel genes, notably KvLQT1 and HERG. Sanguinetti, M. C. et al., Cell (1995) 81:299-307; Sanguinetti, M. C. et al., Nature (1996) 384:80-83; Barhanin J. et al., Nature (1996) 384:78-80; Yang, T. et al., Circ. Res. (1995) 77:1246-1253; McDonald, T. V. et al., Nature (1997) 388:289-292.

The minK (−/−) mice prepared by applicants demonstrated a movement disorder (“tail-chasing”) which has been reported by others who have constructed a similar animal and is thought to represent defective I_{Ks} in the inner ear. Vetter, D. E. et al., Neuron (1996) 17:1251-1264; Drici, M. D. et al., Circ.Res. (1998) 83:95-102.

Electrocardiographic recordings in anesthetized minK (−/−) mice prepared by applicants showed no difference in ECG intervals, including the QT interval, in the genetically modified mice compared to control wild-type animals, whether in neonates or adults. This finding was unanticipated, given the expected expression of minK in the mouse ventricle. However, LacZ staining did reveal that minK was expressed, in a relatively heterogeneous fashion, in mouse atrium, but not ventricle.

The minK (−/−) mice have been used to study the relationship between QT interval and heart rate. This was accomplished under varying levels of anesthesia, and a difference has been identified; however, this experimental approach is potentially seriously compromised by the possibility of a direct anesthetic effect on the QT interval, as well as the heart rate. Until the disclosure of the present invention presented herein, no other characterization of cardiac rhythms in minK (−/−) mice have been attempted.

B. Animal Model of Atrial Fibrillation

In accordance with the present invention, certain minK (−/−) mouse have unexpectedly been determined to exhibit an atrial fibrillation phenotype. Thus, in accordance with the present invention, a method of identifying an animal exhibiting an atrial fibrillation phenotype is provided. The method comprises providing an animal having a minK (−/−) genotype; recording electrocardiograms from the animal provided in step (a) over an extended time period; and (c) identifying an atrial fibrillation pattern in the electrocardiograms to thereby identify the animal as exhibiting an atrial fibrillation phenotype.

In a preferred embodiment, the electrocardiograms (ECGs) from the animal are taken via implantation of a detection device into the peritoneum of the animal and recording the electrocardiograms overthe extended time period while allowing the animal to range freely in a habitat. The detection device is implanted under anesthesia. Once the animal has recovered, ECGs are recorded over an extended period of time while the animal roams around its cage or other habitat or environment. A representative detection device is a telemeter, such as is commercially available from Data Sciences International, Inc. of St. Paul, Minn. Data from the ECGs are analyzed with respect to regularity of rhythm, distribution of heart rates in time and frequency domains, and ECG intervals.

While the following terms are believed to have well defined meanings in the art, the following definitions are set forth to facilitate explanation of the invention.

The term “atrial fibrillation phenotype” is meant to refer to an animal model that expresses an atrial fibrillation phenotype. The term “atrial fibrillation phenotype” is meant to encompass an animal model that exhibits spontaneous atrial fibrillation or to animal model in which atrial fibrillation can be readily induced. Thus, in susceptible animals, such as the minK (−/−) animals disclosed herein, atrial fibrillation can also be elicited by application of a time electrical stimulus to the heart. Atrial fibrillation is meant to have its art-recognized meaning and thus refers to a heart arrhythmia of the atrium.

The term “extended time period” is meant to refer to a period of at least several minutes, and can range to several hours, wherein the minK (−/−) animals can be observed in a free range setting and wherein ECGs from the animals can be monitored to establish the presence of an atrial fibrillation phenotype in the animals. By way of additional example, an “extended time period” in accordance with the present invention can range from about 30 minutes (min) to about 8 hours (hr).

The term “minK (−/−) animal” is meant to refer to an animal that has a minK (−/−) genotype, i.e. the animal has had the endogenous minK gene “knocked out” by homologous recombination or other technique. The term “minK (−/−) animal” is also meant to refer to progeny of such an animal that also have a minK(−/−) genotype. A representative animal is a vertebrate, such as a warm-blooded vertebrate, including mammals and birds. Optionally, the mammal includes, but is not limited to, mouse, dog, pig, rat, ape, monkey, cat, guinea pig, cow, goat and horse. A preferred mammal is a mouse.

A method by which the expression of specific genes can be inhibited in an animal is by genetic manipulations referred to in the art as “gene disruption” or “gene knockout”. Gene knockout is a method of genetic manipulation via homologous recombination that has long been carried out in microorganisms, but has only been practiced in mammalian cells within the past decade. These techniques allow for the use of specially designed DNA molecules (gene knockout constructs) to achieve targeted inactivation (knockout) of a particular gene upon introduction of the construct into a cell. The practice of mammalian gene knockout, including the design of gene knockout constructs and the detection and selection of successfully altered mammalian cells, is discussed in numerous publications, including Thomas et al., (1986) Cell 44(3):419-428; Thomas et al., (1987) Cell 51(3):503-512.; Jasin and Berg, (1988) Genes & Development 2:1353-1363; Mansour et al., (1988) Nature 336:348-352; Brinster, et al., (1989) Proc. Natl. Acad. Sci. USA 86:7087-7091; Capecchi, (1989) Trends in Genetics 5(3):70-76; Frohman and Martin, (1989) Cell 56:145-147; Hasty, et al., (1991) Mol Cell Bio 11(11), pp. 5586-5591; Jeannotte, et al., (1991) Mol Cell Bio 11(11):5578-5585; and Mortensen, et al., (1992) Mol Cell Bio 12(5):2391-2395.

Gene knockouts and gene replacements can be achieved in mammalian zygotes through microinjection techniques well known in the art (Brinster, et al., (1989) Proc. Natl. Acad. Sci. USA 86:7087-7091). The introduction of the DNA constructions used to effect gene knockouts into cultured cells is a more common route to the production of knockout cells, tissues, and organs. In those cases where knockout tissues or organs are desired, cultured embryonic stem cells provide a vehicle to introduce the DNA constructs into cells in culture and to generate animals derived from such engineered cells. Such animals can also be obtained from knockout zygotes obtained by microinjection.

Thus, genetically manipulated animals can be prepared using techniques known in the art. These techniques include, but are not limited to, microinjection, e.g., of pronuclei, electroporation of ova or zygotes, nuclear transplantation, and/or the stable transfection or transduction of embryonic stem cells.

The most well known method for making a genetically manipulated animal, such as a transgenic animal or such as a “knock-out” animal, is that used to produce genetically manipulated mice by superovulation of a donor female, surgical removal of the egg, injection of the gene transcription unit into the pro-nuclei of the embryo, and introduction of the manipulated embryo into the reproductive tract of a pseudopregnant host mother, usually of the same species. See U.S. Pat. No. 4,873,191; Brinster, et al., (1985) Proc. Natl. Acad. Sci. USA 82:4438-4442; Hogan, B., et al., Manipulating the Mouse Embryo, 2nd Ed., Cold Spring Harbor Press, (1994); Robertson, in Robertson (ed.), (1987) Teratocarcinomas and Embryonic Stem Cells a Practical Approach. IRL Press, Eynsham, Oxford, England; Pedersen, et al., (1990) Transgenic Techniques in Mice—A Video Guide. Cold Spring Harbor Laboratory, Cold Spring Harbor, N.Y.

The use of this method to make genetically manipulated livestock is also widely practiced by those of skill in the art. As an example, genetically manipulated swine are routinely produced by the microinjection of nucleic acid molecules into pig embryos. See, for example, PCT Publication No. WO 92/11757.

Another commonly used technique for generating genetically manipulated animal, such as a transgenic animal or such as a “knock-out” animal, involves the genetic manipulation of embryonic stem cells (ES cells) as described in PCT Publication No. WO 93/02188 and Robertson, in Robertson (ed.), (1987) Teratocarcinomas and Embryonic Stem Cells a Practical Approach. IRL Press, Eynsham, Oxford, England. In accordance with this technique, ES cells are grown as described in, for example, Robertson, in Robertson (ed.), (1987) Teratocarcinomas and Embryonic Stem Cells a Practical Approach. IRL Press, Eynsham, Oxford, England, and in U.S. Pat. No. 5,166,065. Genetic material is introduced into the embryonic stem cells by, for example, electroporation according, for example, to the method of McMahon, et al., (1990) Cell 62:1073-1085, or by transduction with a retroviral vector according, for example, to the method of Robertson et al., (1986) Nature 323:445-448, or by any of the various techniques described by Lovell-Badge, in Robertson (ed), (1987) Teratocarcinomas and Embryonic Stem Cells a Practical Approach. IRL Press, Eynsham, Oxford, England.

Chimeric animals are generated as described, for example, in Bradley, in Robertson (ed), (1987) Teratocarcinomas and Embryonic Stem Cells a Practical Approach. IRL Press, Eynsham, Oxford, England. Briefly, genetically modified ES cells are introduced into blastocysts and the modified blastocysts are then implanted in pseudo-pregnant female animals. Chimeras are selected from the offspring, for example by the observation of mosaic coat coloration resulting from differences in the strain used to prepare the ES cells and the strain used to prepare the blastocysts, and are bred to produce non-chimeric genetically manipulated animals.

Other methods for the production of genetically manipulated animals are disclosed in U.S. Pat. No. 5,880,327; U.S. Pat. No. 5,032,407; and PCT Publication No. WO90/08832. The practice of gene knockout in embryonic stem cells, and the generation of engineered animals from such cells, is discussed in numerous publications, including PCT Publication No. WO 93/02188.

Genetically manipulated animals can then be bred according to classical breeding techniques. Such techniques are well known in the art, and are more fully described in U.S. Pat. Nos. 5,633,076 (bovine); 5,573,933 (pigs); 5,675,063 (rabbits); 5,633,425 (mouse); 5,661,016 (mice and other animals) and 4,736,866 (mice and other animals), the entire contents of each of which are herein incorporated by reference.

C. Screening Assays

In another aspect, the present invention pertains to a method of screening substances for their ability to affect atrial fibrillation. A candidate substance is a substance which potentially can affect, modulate or otherwise provide treatment for atrial fibrillation. Thus, a candidate substance identified according to the screening assay described herein has the ability to ameliorate, regulate or otherwise control atrial fibrillation.

Studies in experimental animals as well as in humans suggest that atrial fibrillation can have a number of etiologies. Currently available experimental models include rapid atrial pacing in dogs or other large animals (which after 2-4 weeks does promote atrial fibrillation) and/or the application of action potential-modifying chemicals directly to the atrium, followed by atrial pacing. There is no currently available animal model in which episodes of atrial fibrillation occur spontaneously. Thus, the use of an animal model of atrial fibrillation in accordance with a screening method of the present invention meets a long-felt and continuing need for testing of new drug therapies for this condition.

Representative candidate substances include xenobiotics such as drugs and other therapeutic agents, as well as endobiotics such as steroids, fatty acids and prostaglandins. Other examples of candidate compounds that can be investigated by the screening method of the present invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, and monoclonal antibodies.

In one embodiment of the present invention, the method for screening candidate substances for an ability to modulate atrial fibrillation comprises providing an animal model having an atrial fibrillation phenotype and an minK (−/−) genotype; administering a candidate compound to the animal model; and measuring atrial fibrillation in the animal model to thereby determine the ability of the candidate substance to modulate atrial fibrillation. Preferably, the animal model is prepared according to a process of the present invention as disclosed herein above.

In an alternative embodiment of the present invention, the method of screening candidate substances for an ability to modulate atrial fibrillation comprises: establishing replicate test and control animal models having an atrial fibrillation phenotype and an minK (−/−) genotype; administering a candidate substance to the test animal model but not to the control animal model; measuring cardiac rhythm in the test and the control animal models; and determining that the candidate substance modulates atrial fibrillation based upon a comparison of the cardiac rhythms measured in the test and control animal models.

The animal model can exhibit spontaneous atrial fibrillation. The atrial fibrillation phenotype can also be induced in the animal model via a stimulus to the animal model's heart.

In the screening method of the present invention, the animal model can be a mammal. The mammal can be selected from a group including but not limited to mouse, dog, pig, rat, ape, monkey, cat, guinea pig, cow, goat and horse. Preferably, the mammal is a mouse.

Atrial fibrillation and cardiac rhythms can be measured by recording electrocardiograms from the animal over a predetermined time period. The phrase “predetermined time period” is adopted herein for convenience and is meant to refer to any suitable time period. For example, a predetermined time period can mean an extended time period as defined herein above. Alternatively, the predetermined time period can be a time period suitable for measuring atrial fibrillation after administering a candidate substance to the animal model. The predetermined time period can begin immediately at administration of the candidate substance or can begin at some point subsequent to the administration of the candidate substance, depending on the route of administration and allowing for metabolic processing of the candidate substance in the animal model.

Optionally, the electrocardiograms are recorded over the predetermined time period while allowing the animal to range freely in a habitat. The electrocardiograms from the animal can be taken via surface electrodes or via implantation of detection device into the peritoneum of the animal. A representative detection device for implantation is a telemeter.

The candidate substances can be administered in any suitable manner. For the purposes described above, the identified substances can usually be administered systemically or partially, usually by oral or parenteral administration. The doses to be administered in the screening method of the present invention are determined depending upon age, body weight, symptom, the desired therapeutic effect, the route of administration, the duration of the treatment, and the like.

By way of additional example of approaches for administration of candidate compounds and recordation of ECGs, adult mice are anesthetized with ketamine (30 mg/kg) and pentobarbital (38 mg/kg), as described by Berul et al., Circulation (1996) 94:2641-2648. A jugular vein is cannulated for administration of a candidate compound. Animals are kept on a heating pad set at 38° C. Clips are attached to all 4 limbs, and recordings were obtained with filtering at 3-100 Hz using an analog ECG recorder (E/M VR16). A paper speed of 100 mm/sec is used and tracings are analyzed offline by an investigator blinded to test and control animals using a digitizing tablet and a suitable commercially available analysis program. For each ECG, three consecutive complexes are analyzed.

D. Therapeutic Methods

A therapeutic method is provided in accordance with the method of the present invention. Such a method comprises treating atrial fibrillation in a subject in need of such treatment, by administering to the subject an effective amount of a substance modulates atrial fibrillation in the subject. Such a substance may be identified according to the screening assay set forth above. By the term “modulating”, it is contemplated that the substance can regulate, ameliorate or otherwise control atrial fibrillation.

The subject treated in the present invention in its many embodiments is desirably a human subject, although it is to be understood that the principles of the invention indicate that the invention is effective with respect to other vertebrate species, including mammals, which are intended to be included in the term “subject”. Moreover, a mammal is understood to include any mammalian species in which treatment or prevention of atrial fibrillation is desirable, particularly agricultural and domestic mammalian species.

The methods of the present invention are particularly useful in the treatment of warm-blooded vertebrates. Therefore, the invention concerns mammals and birds.

More particularly, contemplated is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, contemplated is the treatment of livestock, including, but not limited to, domesticated swine (pigs and hogs), ruminants, horses, poultry, and the like.

D.1. Dosage Regimens

Actual dosage levels of active ingredients in the pharmaceutical compositions of this invention may be varied so as to administer an amount of the active compound(s) that is effective to achieve the desired therapeutic response for a particular subject. The selected dosage level will depend upon the activity of the particular compound, the route of administration, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved.

If desired, the effective daily dose may be divided into multiple doses for purposes of administration, e.g., two to four separate doses per day. It will be understood, however, that the specific dose level for any particular subject will depend upon a variety of factors including the body weight, general health, diet, time and route of administration, combination with other drugs and the severity of the particular disease being treated.

The dosage ranges for the administration of a modulator of atrial fibrillation depend upon the form of the modulator, and its potency, as described further herein, and are amounts large enough to produce the desired effect. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

The therapeutic compositions can be administered as a unit dose. The term “unit dose” when used in reference to a therapeutic composition employed in the method of the present invention refers to physically discrete units suitable as unitary dosage for the subject, each unit containing a predetermined quantity of active material calculated to produce the desired therapeutic effect in association with the required diluent; i.e., carrier or vehicle.

The compositions are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies can also be administered.

A therapeutically effective amount is an amount of a modulator sufficient to produce a measurable modulation of atrial fibrillation in a subject being treated, i.e., an atrial fibrillation-modulating amount. The potency of a modulator can vary, and therefore a “therapeutically effective” amount can vary. However, as shown by the present assay methods, one skilled in the art can readily assess the potency and efficacy of a candidate modulator of this invention and adjust the therapeutic regimen accordingly. Thus, modulation of atrial fibrillation can be measured using the screening methods disclosed herein, or by other methods known to one skilled in the art.

For the purposes described above, the identified substances may normally be administered systemically or partially, usually by oral or parenteral administration. The doses to be administered are determined depending upon age, body weight, symptom, the desired therapeutic effect, the route of administration, and the duration of the treatment etc. In a human adult, the doses per person per administration are generally between 1 mg and 500 mg, by oral administration, up to several times per day, and between 1 mg and 100 mg, by parenteral administration up to several times per day. Since the doses to be used depend upon various conditions, as mentioned above, there may be a case in which doses are lower than or greater than the ranges specified above.

D.2. Formulation

The atrial fibrillation modulating substance is adapted for administration as a pharmaceutical composition. Formulation and dose preparation techniques have been described in the art, see for example, those described in Remington's Pharmaceutical Sciences. 17th Ed., 1985. Mack Publishing Company, Philadelphia, Pa, and in U.S. Pat. No. 5,326,902 issued to Seipp et al. on Jul. 5, 1994, U.S. Pat. No. 5,234,933 issued to Marnett et al. on Aug. 10, 1993, and PCT Publication WO 93/25521 of Johnson et al. published Dec. 23, 1993, the entire contents of each of which are herein incorporated by reference.

Solid compositions for oral administration include compressed tablets, pills, dispersible powders, capsules, and granules. In such compositions, one or more of the active substance(s) is or are, admixed with at least one inert diluent (lactose, mannitol, glucose, hydroxypropylcellulose, microcrystalline cellulose, starch, polyvinylpyrrolidone, magnesium metasilicate alminate, etc.). The compositions may also comprise, as is normal practice, additional substances other than inert diluents: e.g. lubricating agents (magnesium stearate, etc.), disintegrating agents (cellulose, calcium glycolate etc.), and assisting agent for dissolving (glutamic acid, aspartic acid, etc.) stabilizing agent (lactose etc.). The tablets or pills may, if desired, be coated with gastric or enteric material (sugar, gelatin, hydroxypropylcellulose or hydroxypropylmethyl cellulose phthalate, etc.). Capsules include soft ones and hard ones.

Liquid compositions for oral administration include pharmaceutically-acceptable emulsions, solutions, suspensions, syrups and elixirs. In such compositions, one or more of the active substance(s) is or are admixed with inert diluent(s) commonly used in the art (purified water, ethanol etc.). Besides inert diluents, such compositions may also comprise adjuvants (wetting agents, suspending agents, etc.), sweetening agents, flavoring agents, perfuming agents and preserving agents.

Other compositions for oral administration include spray compositions which may be prepared by known methods and which comprise one or more of the active substance(s). Spray compositions may comprise additional substances other than inert diluents: e.g. preserving agents (sodium sulfite, etc.), isotonic buffer (sodium chloride, sodium citrate, citric acid, etc.). For preparation of such spray compositions, for example, the methods described in U.S. Pat. Nos. 2,868,691 or 3,095,355 may be used.

Injections for parenteral administration include sterile aqueous or non-aqueous solution, suspensions and emulsions. In such compositions, one or more of active substance(s) is or are admixed with at least one inert aqueous diluent(s) (distilled water for injection, physiological salt solution etc.) or inert non-aqueous diluent(s) (propylene glycol, polyethylene glycol, olive oil, ethanol, POLYSOLBATE 80 (registered trade mark) etc.). Injections may comprise additional other than inert diluents: e.g. preserving agents, wetting agents, emulsifying agents, dispersing agents, stabilizing agents (lactose, etc.), assisting agents such as for dissolving (glutamic acid, aspartic acid, etc.). They may be sterilized, for example, by filtration through a bacteria-retaining filter, by incorporation of sterilizing agents in the compositions or by irradiation. They also be manufactured in the form of sterile solid compositions, for example, by freeze-drying, and which can be dissolved in sterile water or some other sterile diluents for injection immediately before use.

Other compositions for administration include liquids for external use, and endermic linaments (ointment, etc.), suppositories and pessaries which comprise one or more of the active substance(s) and may be prepared by known methods.

### LABORATORY EXAMPLES

The following Laboratory Examples have been included to illustrate preferred modes of the invention. Certain aspects of the following Laboratory Examples are described in terms of techniques and procedures found or contemplated by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be representative only and that numerous changes, modifications and alterations can be employed without departing from the spirit and scope of the invention.

### Example 1

### Preparation of minK (−/−) Mouse

A minK (−/−) mouse was prepared as follows. First, four independent overlapping clones covering ≧20 kb of the minK locus were isolated by screening 10⁶ plaques from a 129 SV genomic library in Lambda Fix II (Stratagene) under high stringency conditions using as a probe the minK cDNA sequence in plasmid T21. Library screening, DNA purification, restriction enzyme mapping and subcloning were performed using standard procedures described by Sambrook, J. et al., Molecular Cloning. A Laboratory Manual, 2nd ed, Cold Spring Harbor Press, (1989).

A targeting vector was constructed using a 6.2 kb Eco RI/Xho I fragment covering exon 2 (which includes the complete coding region of the minK gene described by Honoré, E. et al., EMBO. J. (1991) 10:2805-2811) was subcloned into the Bluescript II KS⁻ vector (Stratagene). The Xba I site was deleted from the polylinker of the vector after digestion by treatment with Klenow and two unique Xba I sites were introduced flanking the minK coding exon, using the Kunkel method of site-directed mutagenesis as described by Kunkel, T. A., Proc. Natl. Acad. Sci. USA (1985) 82:488-492.

The oligonucleotide (5′ CGTCAAGGTT CCCCGGATCT AGAGCAAAAC TCC 3′)(SEQ ID NO:1) used to introduce the upstream site is located 28 bases 5′ of the initiator ATG of the minK gene. The oligonucleotide (5′CCGCTTGTCA CCTCTAGAGT GTGGGGTTCA CGAC 3′)(SEQ ID NO:2) used to introduce the downstream Xba I site is located 11 bases 3′ of the stop codon. Bases which mutate the endogenous minK sequence are underlined. With introduction of these sites, the minK coding region could then be excised in its entirety (total deletion of 427 nt) by digesting the plasmid with Xba I. Re-ligation created a plasmid (pSKII120) that included a unique Xba I site which was then used to accept the lacZ-neo^{r} cassette described below. Consensus splice sites of exon 2 were conserved throughout all manipulations.

The PGK-neo-pA gene was excised from plasmid pPNT (Tybulewicz, V. L., et al. Cell (1991) 65:1153-1163) via NotI/KpnI digestion. In its place, the minK targeting locus from pSKII120 was inserted taking advantage of Not I/Kpn I sites of its flanking polylinker. The resulting plasmid pSKII1147/4 contained the thymidine kinase (TK) gene at the 3′ end.

Concurrently, a cassette was engineered that contained the neomycin resistance (neo^{r}) gene driven by phosphoglycerate kinase-1 (PGK) promoter of pPNT into the vector pSL1180 (Promega). The lacZ gene of pPD46.21, which has an initiator ATG codon and contains a nuclear localization signal (NLS) at the 5′ end, was then added to the 5′ end of the insert. The lacZ-neo^{r} cassette was flanked by Xba I sites which were used for excision of the cassette and its subsequent insertion into the unique Xba I site of pSKII1147/4. This manipulation separated the minK targeting locus into a 4.2 kb long arm and a 1.6 kb short arm homologous to the wild-type (wt) minK locus, and put the lacZ gene under the control of endogenous minK regulatory elements after gene targeting.

Gene targeting was performed as follows. 50 μg of linearized, purified vector DNA was electroporated into 50×10⁸ embryonic stem (ES) cells at 800 V, 3 μF. The TL1 line of ES cells was used. Labosky, P. A. et al., Development 1994; 120:3197-3204. Cells were plated onto irradiated neomycin resistant feeder cells and selected in G418 and ganciclovir. Colonies were harvested and screened using standard procedures described by Hogan, B., et al., Manipulating the Mouse Embryo, 2nd Ed., Cold Spring Harbor Press, (1994).

Genomic Southern analyses were carried out by obtaining DNA from ES cells (Hogan, B., et al., Manipulating the Mouse Embryo, 2nd Ed., Cold Spring Harbor Press, (1994)), digesting the same with Xba I, Southern blotting using standard protocols (Sambrook, J. et al., Molecular Cloning. A Laboratory Manual, 2nd ed, Cold Spring Harbor Press, (1989)) and probed. Mice were genotyped for correct targeting by preparing tail DNA of 3 week old weanlings as described by Hogan, B., et al., Manipulating the Mouse Embryo, 2nd Ed., Cold Spring Harbor Press, (1994). The 5′ probe consisted of a 600 bp restriction fragment that immediately abuts the Xba I site 5′ of the targeting locus. With this probe, hybridization is predicted to an 8 kb Xba I fragment from the wild-type locus and a 5.8 kb fragment for the correctly targeted allele. The 3′ probe consisted of the 400 bp Xho I/Xba I fragment immediately 3′ of the targeting locus. With this probe, the same 8 kb band is expected in the wild type case, while a 2 kb band is expected for the targeted allele.

To verify that there was only a single insertion event per genome, a Southern blot of mouse genomic DNA cut with Eco RI was probed with an internal probe consisting of a 265 bp PCR fragment from the 5′ end of the lacZ gene. A single 7.8 kb fragment is expected, if targeting occurred correctly. Southern blotting of genomic DNA derived from embryonic stem cells selected under G418 and ganciclovir as well as from offspring of chimeras (and later generation) animals demonstrated single inserts of the appropriate size and thus germline transmission.

RNAse protection was performed using standard methods described by Yang, T. et al., Circ. Res. (1995) 77:1246-1253. The riboprobe was complementary to the mouse minK cDNA, lacking the 5′ 70 coding nucleotides. RNAse protection analysis demonstrated that minK mRNA was absent in minK (−/−) animals

Targeted ES cell clones from 129SV mice were microinjected into the blastocoel cavity of embryos derived from natural matings of C57BL/6 mice. The resulting chimeras were bred to two different strains of mice, Black Swiss and 129SV (Taconic). The studies reported here were all conducted using the inbred 129SV mice; however, applicants have observed similar findings with the other strain. Mice were housed in microisolator cages on a 12 hr light/12 hr dark cycle and were specific pathogen free.

### Example 2

### Identification of minK (−/−) Mouse Having Atrial Fibrillation Phenotype

In a series of experiments pertaining to a preferred embodiment of the present invention, minK (−/−) mice were examined for atrial fibrillation by recording heartbeat patterns from a telemeter. The telemeter device was implanted, under anesthesia, and once the mice recovered, hours of ECGs were recorded while the mice ran around their cages. The telemeter was commercially available from Data Sciences International, Inc. of St. Paul, Minn. Data from the ECGs were analyzed with respect to regularity of rhythm, distribution of heart rates in time and frequency domains, and ECG intervals.

Spontaneous episodes of atrial fibrillation, lasting 5 to 150 seconds, were observed in 4 out of 12 minK (−/−) mice. In contrast, only one spontaneous episode lasting less than 3 seconds has been observed in 1/12 wild-type mice. Thus, through the use of the novel method of the present invention which involves recordation of electrocardiograms from free ranging minK (−/−) mice, mice having an atrial fibrillation phenotype were identified.

### Example 3

### Spontaneous and Induced Atrial Fibrillation in minK (−/−) Mice

Applicants have used a minK knockout/lacZ knockin to establish that expression of K+ channel subunit minK is highly restricted (primarily to the sinus and AV nodes and the caudal atrial septum) in mice. Moreover, although sinus rates were similar in anesthetized wild-type (wt) and minK (−/−) animals, significantly slower heart rates were found in the knockouts when an implanted telemetry system was implemented in conscious, unrestrained animals. As discussed herein above, prolonged monitoring (about 30 min to about 8 hr/animal) in minK (−/−) and wt littermates using this system has now revealed long (>5 sec) episodes of atrial fibrillation (AF) in 4/12 (−/−) and in 0/10 wt. Each episode was immediately preceded by heart rate deceleration and/or heart block.

Programmed atrial stimulation was then performed via a 2 Fr esophageal catheter during light ketamine/pentobarbital anesthesia and spontaneous respiration in telemetered animals. There was no significant difference in age, gender, or weight between knockouts and wt littermate controls. AF was elicited by atrial bursts at cycle lengths 20-35 msec in 0/9 wt and 5/11 (−/−) littermates (P<0.03). Episodes lasted 5-150 sec. There was no difference in atrial or AV nodal refractory periods and AV conduction curves were continuous in all animals.

Among knockouts, the mean age of mice with inducible AF was 92±66 days, while the mean among those without arrhythmias was 205±26 days (p=0.02) suggesting age-related changes had a protective effect on atrial susceptibility; there was no gender effect on inducibility. Of the last 10 animals studied, 7 survived the procedure. While it is not applicant's desire to be bound by any theory of operation, it is envisioned that disruption of the spatially restricted expression of minK results in AF both by slowing heart rate and by exaggerating heterogeneity of atrial action potentials.

It is concluded that (1) esophageal pacing can be used to study atrial electrophysiology in mice with a high survival rate; (2) the minK (−/−) mouse is a new model of spontaneous and induced atrial fibrillation; and (3) these data identify abnormal minK function or expression as a contributor to the development of atrial fibrillation.

### References

The references listed below as well as all references cited in the specification are incorporated herein by reference to the extent that they supplement, explain, provide a background for or teach methodology, techniques and/or compositions employed herein.

Barhanin J. et al., Nature (1996) 384:78-80.

Bradley, in Robertson (ed), (1987) Teratocarcinomas and Embryonic Stem Cells a Practical Approach. IRL Press, Eynsham, Oxford, England.

Brinster, et al., (1989) Proc. Natl. Acad. Sci. USA 86:7087-7091.

Brinster, et al., (1985) Proc. Natl. Acad. Sci. USA 82:4438-4442.

Capecchi, (1989) Trends in Genetics 5(3):70-76.

Drici, M. D. et al., Circ.Res. (1998) 83:95-102.

Frohman and Martin, (1989) Cell 56:145-147.

Hasty, et al., (1991) Mol Cell Bio 11(11), pp. 5586-5591.

Hogan, B., et al., Manipulating the Mouse Embryo, 2nd Ed., Cold Spring Harbor Press, (1994).

Honoré, E. et al., EMBO. J. (1991) 10:2805-2811.

Jasin and Berg, (1988) Genes & Development 2:1353-1363

Jeannotte, et al., (1991) Mol Cell Bio 11(11):5578-5585.

Kunkel, T. A., Proc. Natl. Acad. Sci. USA (1985) 82:488-492.

Labosky, P. A. et al., Development 1994;120:3197-3204.

Lovell-Badge, in Robertson (ed), (1987) Teratocarcinomas and Embryonic Stem Cells a Practical Approach. IRL Press, Eynsham, Oxford, England.

Mansour et al., (1988) Nature 336:348-352.

McDonald, T. V. et al., Nature (1997) 388:289-292.

McMahon, et al., (1990) Cell 62:1073-1085.

Mortensen, et al., (1992) Mol Cell Bio 12(5):2391-2395.

PCT Publication No. WO 92/11757

PCT Publication No. WO90/08832

PCT Publication No. WO 93/02188

PCT Publication No. WO 93/02188

PCT Publication WO 93/25521

Pedersen, et al., (1990) Transgenic Techniques in Mice—A Video Guide. Cold Spring Harbor Laboratory, Cold Spring Harbor, N.Y.

Remington's Pharmaceutical Sciences. 17th Ed., 1985. Mack Publishing Company, Philadelphia, Pa.

Robertson, in Robertson (ed.), (1987) Teratocarcinomas and Embryonic Stem Cells a Practical Approach. IRL Press, Eynsham, Oxford, England.

Robertson et al., (1986) Nature 323, pp. 445-448.

Sambrook, J. et al., Molecular Cloning. A Laboratory Manual, 2nd ed, Cold Spring Harbor Press, (1989)

Sanguinetti, M. C. et al., Cell (1995) 81:299-307.

Sanguinetti M. C. et al., Nature (1996) 384:80-83.

Takumi, T., et al., (1988) Science 242:1042-5.

Thomas et al., (1987) Cell 51(3):503-512.

Thomas et al., (1992) Mol Cell Bio 12(7), pp. 2919-2923.

Thomas et al., (1986) Cell 44(3):419-428.

Tybulewicz, V. L., et al. Cell(1991) 65:1153-1163.

U.S. Pat. No. 5,162,215

U.S. Pat. No. 5,573,933

U.S. Pat. No. 5,489,742

U.S. Pat. No. 5,550,316

U.S. Pat. No. 5,166,065

U.S. Pat. No. 4,736,866

U.S. Pat. No. 5,741,957

U.S. Pat. No. 5,880,327

U.S. Pat. No. 5,633,076

U.S. Pat. No. 5,573,933

U.S. Pat. No. 5,625,125

U.S. Pat. No. 5,614,396

U.S. Pat. No. 4,736,866

U.S. Pat. No. 5,633,425

U.S. Pat. No. 5,675,063

U.S. Pat. No. 5,661,016

U.S. Pat. No. 4,873,191

U.S. Pat. No. 5,032,407

U.S. Pat. No. 5,326,902

U.S. Pat. No. 5,234,933

U.S. Pat. No. 2,868,691

U.S. Pat. No. 3,095,355

U.S. Pat. No. 5,648,061

Vetter, D. E. et al., Neuron (1996) 17:1251-1264.

Warmke J. W. et al., Proc. Natl. Acad. Sci. USA (1994) 91:3438-3442.

Yang, T. et al., Circ. Res. (1995) 77:1246-1253.

What is claimed is:

1. A method for identifying an animal exhibiting an atrial fibrillation phenotype, the method comprising:
(a) providing an animal having a minK (−/−) genotype;
(b) recording electrocardiograms from the animal provided in step (a) over an extended time period; and
(c) identifying an atrial fibrillation pattern in the electrocardiograms to thereby identify the animal as exhibiting an atrial fibrillation phenotype.

2. The method of claim 1, where the electrocardiograms are recorded over the extended time period while allowing the animal to range freely in a habitat.

3. The method of claim 1, wherein the electrocardiograms from the animal are taken via implantation of detection device into the peritoneum of the animal.

4. The method of claim 3, wherein the detection device is a telemeter.

5. The method of claim 1, wherein the animal is a mammal.

6. The method of claim 5, wherein the mammal is selected from the group consisting of mouse, dog, pig, rat, ape, monkey, cat, guinea pig, cow, goat and horse.

7. The method of claim 6, wherein the mammal is a mouse.

8. An animal exhibiting an atrial fibrillation phenotype identified according to the method of claim 1.

9. A non-human animal having an minK (−/−) genotype and an atrial fibrillation phenotype.

10. The non-human animal of claim 9, wherein the animal is a mammal.

11. The non-human animal of claim 10, wherein the mammal is selected from the group consisting of mouse, dog, pig, rat, ape, monkey, cat, guinea pig, cow, goat and horse.

12. The non-human animal of claim 11, wherein the mammal is a mouse.

13. A method for screening candidate substances for an ability to modulate atrial fibrillation, the method comprising:
(a) providing an animal model having an atrial fibrillation phenotype and a minK (−/−) genotype;
(b) administering a candidate compound to the animal model; and
(c) measuring atrial fibrillation in the animal model to thereby determine the ability of the candidate substance to modulate atrial fibrillation.

14. The method of claim 13, wherein the animal model is a mammal.

15. The method of claim 14, wherein the mammal is selected from the group consisting of mouse, dog, pig, rat, ape, monkey, cat, guinea pig, cow, goat and horse.

16. The method of claim 15, wherein the mammal is a mouse.

17. The method of claim 13, wherein the atrial fibrillation phenotype further comprises a spontaneous atrial fibrillation phenotype.

18. The method of claim 13, wherein the atrial fibrillation phenotype is induced in the animal model via a stimulus to the animal model's heart.

19. The method of claim 13, wherein atrial fibrillation is measured by recording electrocardiograms from the animal over a predetermined time period.

20. The method of claim 19, where the electrocardiograms are recorded over the predetermined time period while allowing the animal to range freely in a habitat.

21. The method of claim 17, wherein the electrocardiograms from the animal are taken via implantation of detection device into the peritoneum of the animal.

22. The method of claim 21, wherein the detection device is a telemeter.

23. A method of screening candidate substances for an ability to modulate atrial fibrillation, the method comprising:
(a) establishing replicate test and control animal models having an atrial fibrillation phenotype and a minK (−/−) genotype;
(b) administering a candidate substance to the test animal model but not to the control animal model;
(c) measuring cardiac rhythm in the test and the control animal models; and
(d) determining that the candidate substance modulates atrial fibrillation based upon a comparison of the cardiac rhythms measured in the test and control animal models.

24. The method of claim 23, wherein the animal is a mammal.

25. The method of claim 24, wherein the mammal is selected from the group consisting of mouse, dog, pig, rat, ape, monkey, cat, guinea pig, cow, goat and horse.

26. The method of claim 23, wherein the mammal is a mouse.

27. The method of claim 23, wherein the atrial fibrillation phenotype further comprises a spontaneous atrial fibrillation phenotype.

28. The method of claim 23, wherein the atrial fibrillation phenotype is induced in the animal model via stimulus to the animal model's heart.

29. The method of claim 23, where the cardiac rhythms in the test and control animal models are measured by recording electrocardiograms from the animal models over a predetermined time period.

30. The method of claim 29, where the electrocardiograms are recorded over the extended time period while allowing the animal to range freely in a habitat.

31. The method of claim 29, wherein the electrocardiograms from the animal are taken via implantation of detection device into the peritoneum of the animal.

32. The method of claim 31, wherein the detection device is a telemeter.

33. A method of modulating atrial fibrillation in a vertebrate, the method comprising administering to a vertebrate in need thereof an effective amount of a substance capable of modulating atrial fibrillation in the vertebrate, whereby modulation of atrial fibrillation is accomplished.

34. The method of claim 29, wherein the vertebrate is a mammal.
